(19) United States
(12) Patent Application Publication
(10) Pub. No.: US 2009/0149884 A1
(43) Pub. Date: Jun. 11, 2009

(54) SYSTEM AND METHOD FOR BRIDGE ANCHOR TENDON ATTACHMENT

(75) Inventors: Stephen J. Snyder, Encino, CA (US); Nathan B. Snyder, Los Angeles, CA (US); George J. Rohlinger, Coeur d'Alene, ID (US); M. Mary Sinnott, Logan, UT (US)

Correspondence Address: MEDICINELODGE INC., 124 South 600 West, LOGAN, UT, 84321, US

(73) Assignee: REDYNS MEDICAL, LLC, Los Angeles, CA (US)

(21) Appl. No.: 12/184,779

(22) Filed: Aug. 1, 2008

Related U.S. Application Data
(60) Provisional application No. 60/963,081, filed on Aug. 2, 2007.

(51) Int. Cl. A61B 17/04

(52) U.S. Cl. 606/233; 606/228; 606/232

(57) ABSTRACT

A tendon attachment system may include two fasteners forming a fastener assembly. The first fastener may comprise a capture body with suture apertures and a threadable capture body hole, two sharpened legs attached to the capture surface, and a tendon channel. The suture may be secured to a tendon, and slid through the apertures guiding the first fastener to the fixation site. The two sharpened legs may be driven into the bone. The capture body may provide security of the tendon to the bone and the sutures may be secured to the first fastener. The second fastener may comprise a head portion, and a threadable portion which may pass through the capture body hole and threadably engage with the bone, further advancing the first fastener toward the bone. The suture may be further secured by the engagement of the second fastener to the first fastener.

### CROSS-REFERENCE TO RELATED APPLICATIONS

This application claims the benefit of the following, which is incorporated herein by reference:

Pending prior U.S. Provisional Patent Application No. 60/963,081, filed 2 Aug. 2007, which carries Applicants' docket no. RED-3 PROV, and is entitled Surgical Implant and Technique.

### BACKGROUND OF THE INVENTION

1. The Field of the Invention

The present invention relates to generally to the attachment of tendon to bone, and more particularly, to systems and methods for implantable bridge anchors for tendon attachment to bone.

2. The Relevant Technology

One of the most difficult aspects of a tendon attachment procedure is providing a consistency and superior fixation to the bone. While this challenge exists in the re-attachment of to any tendon to the corresponding bone, it represents a particular problem for biceps tendon re-attachment (biceps tenodesis). In some cases the biceps tendon is not fixed to the bone but is simply cut allowing the muscle to retract distally. Although this approach can be effective it creates a “Popeye” deformity. In one method of biceps tenodesis the biceps stump is secured to the rotator cuff. Since this is a soft tissue fixation it is inherently weak.

Other techniques include the “Key-Hole” technique in which the tendon is rolled into a ball and secured inside a key-hole shaped cavity in the bicipital groove. This procedure requires deltoid split and surgical exposure. Furthermore the key-hole requires a large hole in the bone and it is difficult to secure the tendon in the hole.

Yet another technique is referred to as the tunnel technique where the tendon is cut from the superior labral attachment and secured to the proximal humerus via sutures passing through two holes that are drilled into the humerus. This method requires a mini-open incision in the axilla and risks regional nerve damage.

Yet another technique uses a suture anchor to secure the tendon to the humerus. Primary drawbacks of this technique include the anchor dislodging from the driver, anchor pullout, suture cutting through the degenerative tendon, and difficult arthroscopic visualization.

Yet another technique, known as the interference screw technique, requires a large hole to be drilled into the bone. A screw is secured to the tendon and then the screw is secured into the bone over the top of the tendon. However, this technique creates a large socket and may result in development of a cyst. Furthermore, this technique requires extra-corporeal extraction of the tendon, i.e., the drawing of the tendon out of the body. This adds to the time required for surgery, and to the probability of damage or infection to the surrounding tissues.

As the above described techniques illustrate, the existing systems and procedures for attaching a tendon to a bone may not be as effective as desired or may lead to further reconstructive surgeries.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1 illustrates a front view of a fastener assembly with a suture, a first fastener and a second fastener;

FIG. 2 illustrates a perspective view of a suture, a first fastener comprising a first sharpened leg distal portion, a capture body proximal portion, a second sharpened leg distal portion, a first aperture, a second aperture, a first fastener proximal hole and a tendon channel;

FIG. 3 illustrates an exploded perspective view of the fastener assembly of FIG. 1 with a bone and a tendon, the second fastener is cannulated comprising a proximal head, and a threaded distal portion;

FIG. 4 illustrates a cross-sectional view of the first fastener of FIG. 1;

FIG. 5 illustrates a top rotated view of the first fastener of FIG. 1;

FIG. 6 illustrates a perspective view of an impactor interfaced with the first fastener of FIG. 1, the impactor comprising a threadable distal end first fastener interface, an impactor body, suture grooves at the distal end, a K-wire hole through the longitudinal center, and a flat proximal surface;

FIG. 7 illustrates a side view of the impactor without the first fastener attachment;

FIG. 8 illustrates a cross sectional side view of the impactor without the first fastener attachment;

FIG. 9 illustrates a section view of the fastener assembly with the first fastener, the second fastener showing the driving of the second fastener into a bone with a screwdriver using a K-wire as a guide;

FIG. 10 illustrates a perspective view of a screwdriver with a screw driver body, a distal end second fastener interface, and a K-wire hole through the longitudinal center;

FIG. 11 illustrates a perspective view of a fastener to a bone, which may be a humerus bone;

FIG. 12 illustrates a perspective view of an alternative embodiment of a first fastener comprising a first sharpened leg distal portion, a capture body proximal portion, a first aperture, a second aperture, and a first fastener proximal hole;

FIG. 13 illustrates a perspective view of an alternative embodiment of a first fastener comprising a first sharpened leg distal portion, a capture body proximal portion, a first aperture, a second aperture, a first fastener proximal hole, a sharpened prong distal portion, and a tendon channel; and

FIG. 14 illustrates a perspective view of an alternate embodiment of a first fastener comprising a first sharpened leg distal portion, a capture body proximal portion, a second sharpened leg distal portion, a first aperture, a second aperture, a first fastener proximal hole, a tendon channel, lateral barbs and medial barbs.

### DETAILED DESCRIPTION OF THE PREFERRED EMBODIMENTS

The present invention relates to systems and methods for fastener assemblies for securing a tendon. Those of skill in the art will recognize that the following description is merely illustrative of the principles of the invention, which may be applied in various ways to provide many different alternative embodiments. This description is made for the purpose of illustrating the general principles of this invention and is not meant to limit the inventive concepts in the appended claims.

One embodiment of the present invention includes a suture, a first fastener with a sharpened end and a capture body with an at-least partially spherical shaped head, an impactor, a second fastener with a sharpened end and an at-least spherical shaped head, which may be a bone screw, and a screw driver. A method of securing a tendon to a bone consists of securing a suture to a tendon proximately to a fixation site on the bone and passing a suture through a first aperture site on the first fastener. This suture through the first aperture acts as a guide for the fastener toward the fixation site on the bone. The first fastener penetrates the bone compressing the tendon against the fixation site and the suture is secured to the first fastener. The second fastener penetrates the bone piercing the first fastener hole and first fastener channel, potentially piercing the tendon as well, adding further securement of the tendon to the bone as well as further securement of the suture to the first fastener.

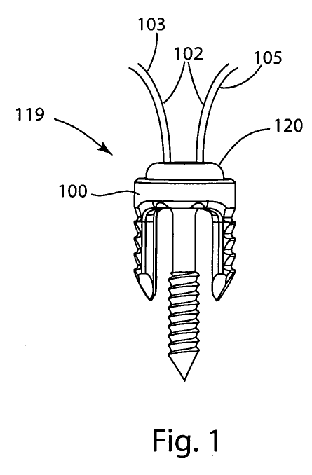

FIGS. 1-5 illustrate an embodiment of a tendon fastener assembly with a suture, a first fastener with two sharpened ends, an impactor and a second fastener, which may be a bone screw, and a screw driver.

Referring to FIG. 1, tendon fastener assembly 119 includes a suture 102 comprising a first suture working portion 103 and a second suture working portion 105, a first fastener 100 and a second fastener 120.

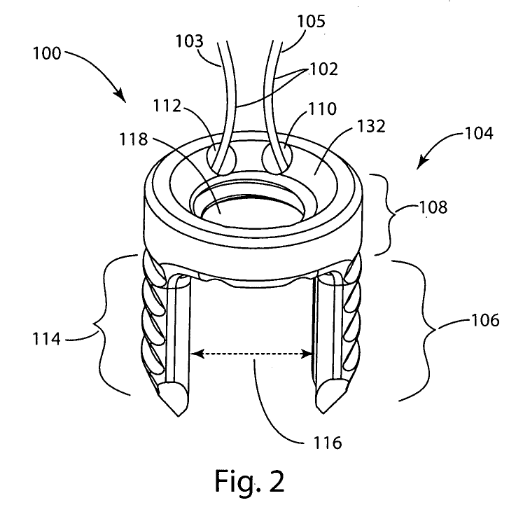

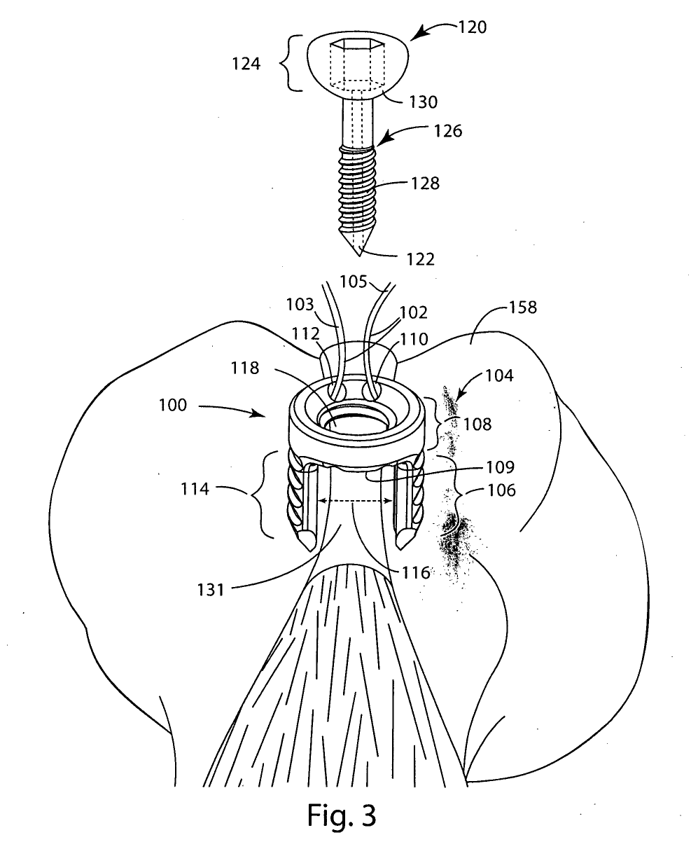

Referring to FIG. 2, the first fastener 100 comprises a first sharpened leg 106, which may taper gradually to a sharpened point at the distal end of the first sharpened leg or may taper abruptly to a sharpened point at the distal end of the first sharpened leg, and a second sharpened leg 114, which may taper in the same manner as the first sharpened leg, which extend from opposite sides of a capture body 108. Each sharpened leg may be separate and independently insertable into the bone. The sharpened legs can be connected by a flexible member that permits independent insertion of screws. The capture body 108 is generally circular and includes a rim 132, through which a first aperture 110 and a second aperture 112 extend. The first and second apertures 110, 112 may be on one side of the capture body 108, meaning only two apertures or an aperture set, or may be on both sides, the second aperture set opposite the first aperture set. The presence of two of each of the apertures 110, 112 enables a suture working portion to be drawn through multiple holes, or enables the capture body 108 to receive four independent suture working portions (not shown). This facilitates the use of the first fastener 100 with a variety of hitches used to attach the suture 102 to the tendon 131. FIGS. 2 and 3 illustrate the use of only first and second suture working portions 103, 105 for clarity; however, consistently with the foregoing, two additional suture working portions (not shown) may optionally extend through the additional first and second apertures 110, 112.

A first fastener proximal hole 118 extends through the capture body 108 and may be an engagement feature for a second fastener 120. The gap between the first sharpened leg 106 and the second sharpened leg 114 forms a channel 116 sized to receive at least a portion of a tendon. Each aperture 110, 112 is sized to slidably receive one suture working portion 103, 105. An alternate embodiment may have one aperture on one side or both sides of the first fastener 100 sized to slidably receive two working portions 103,105, or alternatively, one or any other number of working portions.

Alternatively the first fastener 100 capture body 108 may have alternate shapes such as square, rectangular, hexagonal or other common shapes known or used in the art, with the rim 132 of the capture body 108 configured to engage the second fastener 120. The apertures 110, 112 are configured in a similar manner to the circular embodiment of the first fastener 100 in that they may be positioned on one side of the capture body 108 or on both sides of the capture body, the second aperture set opposite the first aperture set. Furthermore, the apertures 110, 112 may be positioned around the entire rim 132 equidistant to each other.

Referring to FIG. 3, the suture 102 is secured to a tendon 131 using a Mehalik Hitch, or other method known in the art, proximate to a fixation site on a bone 158, which may be a humerus bone. Applying a hitch to the tendon leaves two working portions of suture. The first aperture 110 is sized to slidably receive one working portion 103. The first working portion 103 is slid through the first aperture 110 and the second working portion 105 is slid through the second aperture 112 and may thereby guide the first fastener 100 to the fixation site on the bone 158.

As the first fastener 100 is guided toward the fixation site, the tendon 131 is captured in the channel 116 between the sharpened legs 106, 114. The first sharpened leg 106 is driven into the bone 158 at the fixation site to a point where the capture surface 109 compresses the tendon 131 to the bone 158. The second sharpened leg 114 is driven into the bone 158 further compressing the tendon 131 to the bone 158.

The first fastener proximal hole 118 is sized to receive the second fastener 120. The second fastener 120 may be a bone screw, a nail, or other possible fasteners well known in the art. The proximal end of the second fastener 120 comprises a head 124 and the distal portion 126 comprises a sharpened point 122, a threaded portion 128, and may have a cannulation running longitudinal through the center of the second fastener 120. The threaded portion 128 may extend the entire length of the distal portion 126 of the second fastener. The head 124 includes a screw driver engagement portion which may be hexagonal, square, star-shaped or have any other shape known in the art for engaging a screw driver with the same engagement feature.

The second fastener 120 is secured by sliding the distal portion 126 through the first fastener proximal hole 118 into the channel 116 using a screwdriver 150 or other method well known in the art, thus joining the first fastener 100 and second fastener 120 while driving the second fastener 120 into the bone 158. The sharpened point 122 and threaded portion 128 may pierce the captured tendon 131 and further secure it to the bone 158. Alternatively, the threaded portion 128 need not pierce the captured tendon 131, but may instead pass alongside it. Further advancement of the sharpened distal end 126 of the second fastener 120 urges the first fastener 100 toward the bone 158.

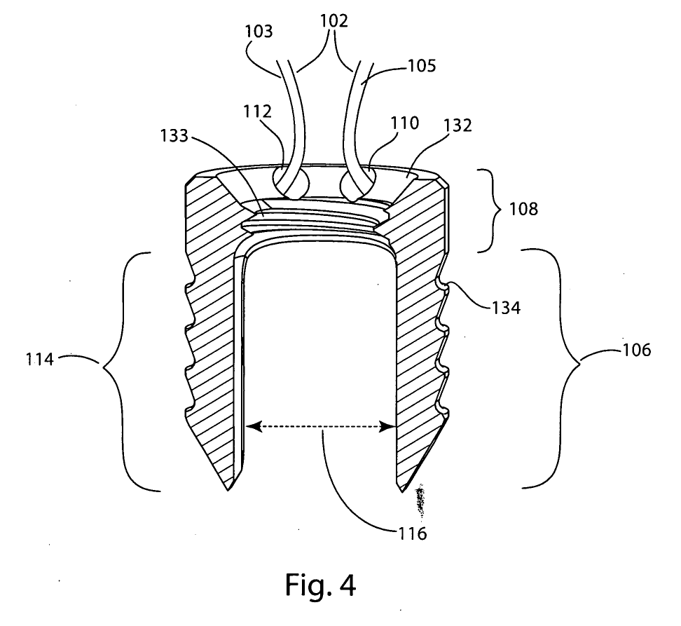

Referring to FIG. 4, barbs 134 may extend from the first fastener 100 first sharpened leg 106 or may extend from the first fastener 100 second sharpened leg 114 or both for further securability of the first fastener 100 to the bone 158.

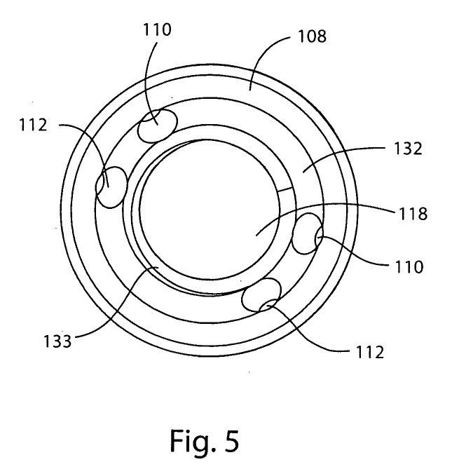

Referring to FIGS. 4 and 5, the rim 132 on the first fastener 100 is contoured to receive the distal portion 130 of the head 124 of the second fastener 120, thus allowing the first fastener 100 and second fastener 120 to engage and become secure to each other. The capture body 108 may include threads 133 which are shaped to engage with an impactor tool configured to drive the first fastener 100 into a bone. The interface between the rim 132 and distal end 130 of the head 124 of the second fastener 120 increases suture 102 securability further by trapping and locking the suture 102 between the first fastener 100 and second fastener 120.

Alternative embodiments for securing the second fastener to the first fastener may include a taper, a snap fit, a cam lock, a separate lock out fastener secured to the fastener assembly, a threaded head portion of the second fastener or any other feature known in the art.

The fastener assembly 119 may be comprised of any materials commonly surgically implanted including but not limited to stainless steels, titanium and its alloys, cobalt-chrome and its alloys, superelastic alloys, shape memory alloys, ceramics, natural or artificial bones, porous coatings, porous materials, porous substrates, bioactive materials such as hydroxylapatite, polyesther, silk, bioabsorbable materials, and polymers such as polyethylene, polyetheretherketone (PEEK), polyaryletherketone (PAEK), polylactic acid (PLA), and acetal copolymer.

It is important to note that the system of attaching the tendon 131 to the bone 158 with the first fastener 100 can operate with or without the second fastener 120.

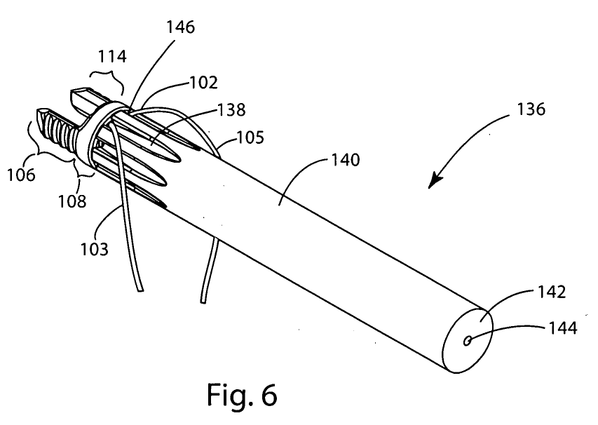

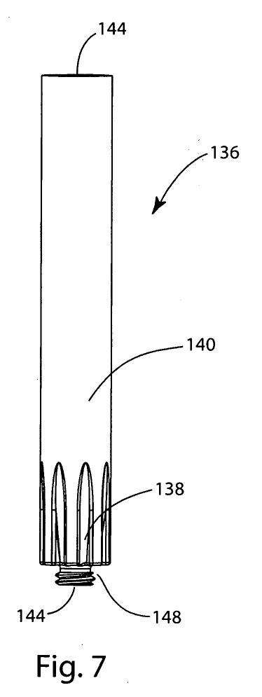

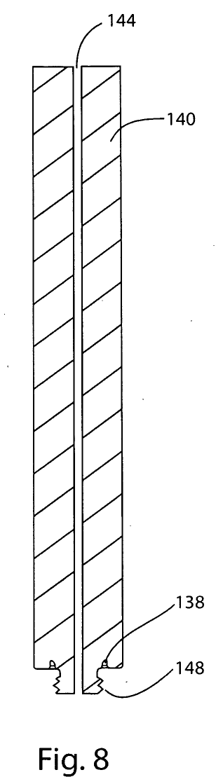

FIGS. 6 through 10 illustrate one method of implementing the present invention to attach a tendon to a bone. Referring to FIG. 6, an impactor 136 is engaged with the first fastener 100 at an engagement interface 146. Referring also to FIGS. 7 and 8, the impactor 136 is comprised of suture grooves 138, an impactor body 140, a flat proximal portion 142, a K-wire aperture 144 running longitudinally through the center of the impactor 136, and a threadable engagement feature 148. The suture grooves 138 allow for pass through of the two suture working portions 103, 105 after passing through the first and second apertures 110,112 of the first fastener 100 proximal end capture body 108.

The threadable engagement feature 148 is threaded into the threads 133 encircling the proximal hole 118 of the first fastener 100. The suture 102 may be secured to a tendon 131 using a Mehalik Hitch, or other method known in the art, thus providing two suture working portions 103, 105. The suture working portions 103, 105 pass through the first and second apertures 110, 112 of the proximal end 108 of the first fastener 100 and may further pass through the suture grooves 138 on the impactor 136. The suture working portions 103, 105 provide a guide for the first fastener 100 and the impactor 136 to a fixation point on the bone 158. The impactor 136 is used to drive the first fastener 100 into the bone. A K-wire may pass through the center of the impactor 136 K-wire aperture 144 and may pass through the center of the first fastener 100 proximal hole 118. The K-wire may be driven into the bone 158 at the fixation point. The impactor 136 may be disengaged from the first fastener 100 by unthreading the threadable engagement feature 146 from the proximal hole 118 of the first fastener and then withdrawn over the K-wire, leaving the K-wire extending from the bone 158 through the first fastener 100. The suture 102 may be secured to the first fastener 100.

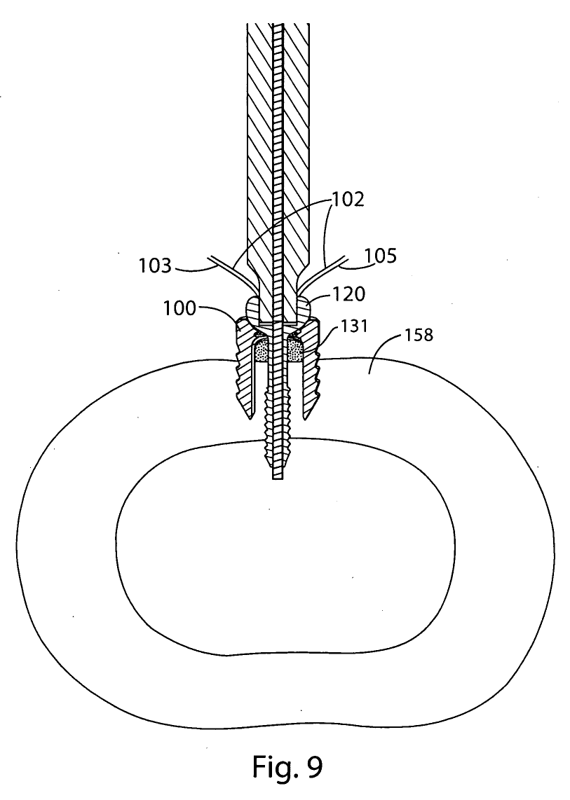

Referring to FIG. 9, the K-wire may be passed through the center of the second fastener 120 and used to guide the second fastener along the K-wire to the first fastener 100. The second fastener 120 may be threaded through the proximal hole 118, but the proximal hole 118 may also slidably receive the second fastener 120.

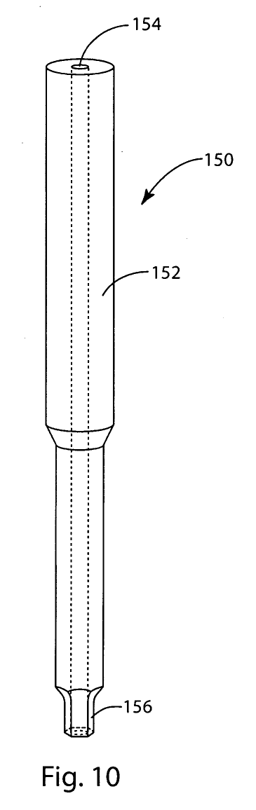

Referring to FIG. 10, a screwdriver 150 may slide over and encircle the K-wire and engage the second fastener 120 to secure the second fastener 120 to the bone 158. The screwdriver 150 comprises a screw driver body 152, a K-wire aperture 154 that extends longitudinally the length of the screwdriver, and a second fastener engagement feature 156. The second fastener engagement feature 156 engages the proximal end 124 of the second fastener 120. The screwdriver 150 drives the second fastener 120 slidably or threadably through the proximal hole 118 of the first fastener 100 and the second fastener 120 pierces the tendon channel 116. The screwdriver 150 further threadably drive the second fastener 120, which may pierce the tendon 131, into the bone 158, further securing the first fastener 100 and further securing the suture 102 against the distal portion 130 of the proximal end 124 of the second fastener to the rim 132 of the first fastener 100. The screwdriver 150 is disengaged from the proximal end 124 of the second fastener 120 and the screwdriver 150 may be withdrawn from encircling the K-wire.

An alternative embodiment of the second fastener may be a nail (not shown), which may comprise a sharpened distal portion, a proximal head portion and a K-wire hole passing longitudinally through the nail. The nail may further comprise an engagement feature designed to engage a nail impactor. The nail impactor may comprise a body, a K-wire hole passing longitudinally through the nail impactor body and an engagement feature designed to engage the nail.

One method for attaching the second fastener nail to the bone may involve sliding the nail and nail impactor over the K-wire so it is encircling the K-wire. The second fastener nail passes slidably through the proximal hole of the first fastener piercing the tendon channel. The nail impactor may drive the nail, which may pierce the tendon, into the bone further securing the first fastener and the suture. The nail impactor may be disengaged from the proximal head portion and the nail impactor may be withdrawn from encircling the K-wire.

Alternative embodiments of a fastener known in the art that may be used in place of the second fastener described above to provide further securability of the tendon to the bone, further securability of the suture to the first fastener and/or further securability of the second fastener to the first fastener.

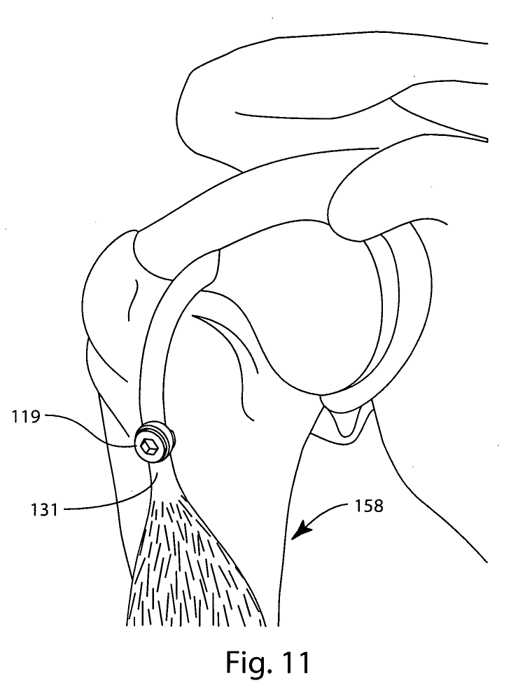

Referring to FIG. 11, a complete fastener assembly 119 is securing a tendon 131 to a bone 158, which may be a humerus bone. The K-wire and tools have been disengaged and removed, and the suture working portions 103, 105 have been cut.

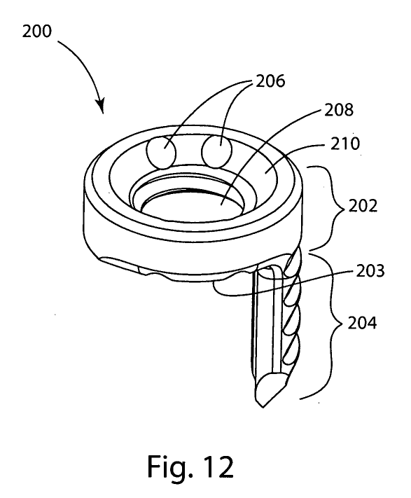

FIGS. 12-14 illustrate alternate embodiments of a first fastener with suture apertures and configured to receive a second fastener through a proximal hole in the first fastener. It is important to note that a system of attaching a tendon to a bone with a first fastener in each of the following embodiments can operate with or without a second fastener.

Referring to FIG. 12, an alternative embodiment of the first fastener with a single sharpened distal leg is shown. A first fastener 200 is shown comprising a proximal end capture body 202 with suture apertures 206, a proximal end hole 208 and a single sharpened distal leg 204, which may taper gradually to a sharpened point at the distal end of the single sharpened leg or may taper abruptly to a sharpened point at the distal end of the single sharpened leg. The capture body 202 comprises a rim 210, wherein the apertures 206 are located on the rim 210 of the proximal end. The capture body 202 further comprises a proximal end hole 208 wherein the impactor 136 may be engaged and through which the second fastener 120 may be threaded to engage with the bone 158.

The features of the FIG. 12 embodiment mirror the preferred embodiment of FIGS. 1-10 with the exception that the FIG. 12 embodiment features a single sharpened distal leg 204 for the first fastener 200. The features for tendon capture, suture attachment and first fastener and second fastener bone engagement remain unchanged from the previous embodiment.

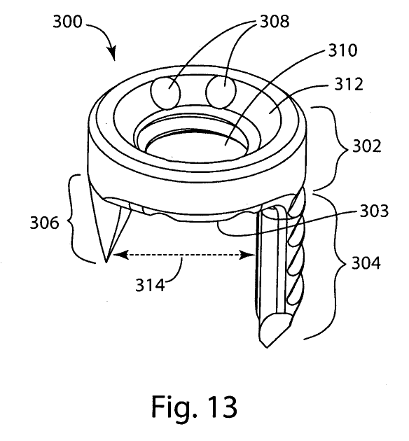

Referring to FIG. 13, an alternative embodiment of a first fastener with a single sharpened distal leg, which may taper gradually to a sharpened point at the distal end of the single sharpened leg or may taper abruptly to a sharpened point at the distal end of the single sharpened leg, and a single sharpened distal prong is shown. A first fastener 300 is shown comprising a proximal end capture body 302 with a rim 312 wherein suture apertures 308 are located; a proximal end hole 310, a single sharpened distal leg 304 extending from the capture body 302, and a sharpened prong 306 extending from the capture body 302 located opposite the sharpened distal leg 304. The space between the sharpened prong 306 and sharpened distal leg 304 forms a tendon channel 314.

The features of the FIG. 13 embodiment mirror the preferred embodiment of FIGS. 1-10 with the exception that the FIG. 13 embodiment features a single sharpened distal leg 304 and a single sharpened prong 306 located opposite the sharpened distal leg. The features for tendon capture, suture attachment and first fastener and second fastener bone engagement remain unchanged from the previous embodiment.

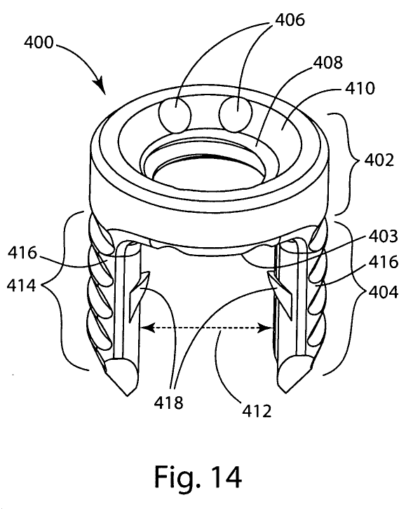

Referring to FIG. 14, an alternative embodiment of a first fastener with two sharpened distal legs with both lateral and medial barbs is shown. A first fastener 400 is shown comprising a proximal end capture body 402 with a rim 410 wherein suture apertures 406 are located, a proximal end hole 408, a first sharpened distal leg 404, which may taper gradually to a sharpened point at the distal end of the first sharpened leg or may taper abruptly to a sharpened point at the distal end of the first sharpened leg, a tendon channel 412, and a second sharpened distal leg 414, which may taper in the same manner as the first sharpened leg. Lateral barbs 416 are located on the sharpened distal legs 404, 414, and at least one medial barb 418 is located on one or both of the sharpened distal legs 404, 414.

The features of the FIG. 14 embodiment mirror the preferred embodiment of FIGS. 1-10 with the exception that the FIG. 13 embodiment features a medial barb 418 for further securability of the first fastener 400 to the bone 158. The first sharpened distal leg 404 is driven into the bone 158 at the fixation site to a point where the capture body 402 compresses the tendon 131 to the bone 158. The second sharpened distal leg 414 is driven into the bone 158 further compressing the tendon 131 to the bone 158.

Similar to the preferred embodiment the lateral barbs 416 provide further securability of the first fastener 400 to the bone 158. The medial barbs 418 add even further securability of the first fastener 400 when the second fastener 120 drives the first fastener 400 further into the bone, which may cause the first and second sharpened distal legs 404, 414 to wedge, further engaging the medial barbs 418 adding greater first fastener 400 and tendon security.

Another possible embodiment of the first fastener may be similar to FIG. 12; a first fastener (not shown) comprised of a proximal end capture body and a single sharpened distal leg. However, the capture body comprises apertures on a rim sized to slidably receive suture working portions and a proximal end hole on the capture body, sized to slidably or threadably receive the second fastener, located off center wherein the second fastener may act as a second sharpened distal leg.

A further embodiment of the first fastener may be a first fastener (not shown) comprising a proximal end capture body and a distal end first sharpened leg extending from the capture body. The first sharpened leg may comprise a backward facing hook or barb extending from the distal end of the first sharpened leg providing added securability of the first fastener to the bone. A second sharpened leg may extend from the capture body opposite the first sharpened leg and may also comprise a backward facing hook or barb extending from the distal end of the second sharpened leg. Each sharpened leg may be separate and independently insertable into the bone. The sharpened legs can be connected by a flexible member that permits independent insertion of screws.

Fastener assemblies 200, 300, and 400, as well as other embodiments, may be assembled and implanted similarly to the method described for first fastener assembly 100. In all assemblies the suture is secured to the tendon with the sutures then being passed through the suture apertures in the first fastener. The sutures serve as a guide to the fixation point for the first fastener. The first fastener is driven into the bone with an impactor which is engaged to the first fastener. The K-wire is driven into the bone through the impactor. The impactor is disengaged and the suture is secured to the first fastener.

The second fastener and screwdriver are guided with the K-wire to the first fastener and fixation site. The second fastener pierces the first fastener hole and is driven into the bone with the screwdriver, piercing the tendon. The second fastener further secures the first fastener and the tendon to the bone. The second fastener further secures the suture to the first fastener as the second fastener engages the rim of the first fastener. The system of attaching a tendon to a bone with a first fastener can operate with or without a second fastener.

The present invention may be embodied in other specific forms without departing from its spirit or essential characteristics. For example, above are described various alternative examples of fasteners for providing tendon attachment. It is appreciated that various features of the above-described examples can be mixed and matched to form a variety of other combinations and alternatives. It is also appreciated that this system should not be limited to a single tendon and bone. This fastener system may be used to attach any tendon to its appropriate bone. As such, the described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A system for securing a tendon to a bone, the system comprising:
a suture; and
a first fastener comprising:
a distal portion comprising a first sharpened leg shaped to penetrate the bone; and
a proximal portion extending nonparallel to the first sharpened leg such that the proximal portion defines a capture surface capable of compressing the tendon against the bone as the first sharpened leg penetrates the bone, the proximal portion defining a first aperture sized to slidably receive the suture.

2. The system of claim 1, wherein the proximal portion further defines a second aperture sized to slidably receive the suture.

3. The system of claim 1, wherein the distal portion of the first fastener further comprises a second sharpened leg shaped to penetrate the bone, wherein the first and second sharpened legs extend from opposite sides of the capture surface to define a channel sized to receive the tendon.

4. The system of claim 1, further comprising a second fastener comprising a sharpened distal end shaped to penetrate the bone, and a proximal end securable to an engagement feature of the proximal portion of the first fastener.

5. The system of claim 4, wherein the second fastener comprises a bone screw, wherein the proximal end comprises a head and the engagement feature comprises a hole sized to receive the sharpened distal end, wherein the hole is bounded by a rim against which the head seats to enable the bone screw to urge the first fastener toward the bone in response to advancement of the sharpened distal end into the bone.

6. The system of claim 5, wherein the engagement feature comprises threads bounding the hole to threadably receive threads of the sharpened distal end.

7. The system of claim 5, wherein the distal portion of the first fastener further comprises a second sharpened leg shaped to penetrate the bone, wherein the first and second sharpened legs extend from opposite sides of the capture surface to define a channel sized to receive the tendon, wherein the hole passes through the capture surface such that the sharpened distal end of the second fastener pierces the channel in response to advancement of the second fastener through the hole.

8. A method for securing a tendon to a bone, the method comprising:
securing a suture to the tendon proximate a fixation site on the bone;
passing a first working portion of the suture through a first aperture of a first fastener;
sliding the first working portion through the first aperture to guide the first fastener toward the fixation site; and
penetrating the bone of the fixation site with a first sharpened leg of the first fastener such that a capture surface defined by a proximal portion of the first fastener compresses the tendon against the fixation site.

9. The method of claim 8, wherein the first fastener comprises a second aperture, the method further comprising sliding a second working portion of the suture through the second aperture to further guide the first fastener toward the fixation site.

10. The method of claim 8, further comprising penetrating the bone of the fixation site with a second sharpened leg of the first fastener, wherein the first and second sharpened legs extend from opposite sides of the capture surface to define a channel, the method further comprising receiving the tendon within the channel.

11. The method of claim 8, further comprising:
penetrating the bone with a sharpened distal end of a second fastener; and
securing a proximal end of the second fastener to an engagement feature of the proximal portion of the first fastener.

12. The method of claim 11, further comprising piercing the tendon with the sharpened distal end prior to penetration of the bone by the sharpened distal end.

13. The method of claim 11, wherein the second fastener comprises a bone screw, wherein the proximal end comprises a head and the engagement feature comprises a hole, wherein the hole is bounded by a rim, the method further comprising:
receiving the sharpened distal end within the hole; and
seating the head against the rim such that further advancement of the sharpened distal end into the bone urges the first fastener toward the bone.

14. The method of claim 13, wherein the engagement feature comprises threads bounding the hole, wherein receiving the sharpened distal end within the hole comprises threadably receiving threads of the sharpened distal end with the threads of the engagement feature.

15. The method of claim 13, further comprising penetrating the bone of the fixation site with a second sharpened leg of the first fastener, wherein the first and second sharpened legs extend from opposite sides of the capture surface to define a channel, wherein the hole passes through the capture surface, the method further comprising:
receiving the tendon within the channel; and
advancing the second fastener through the hole to pierce the tendon with the sharpened distal end.

16. The method of claim 8, further comprising securing the suture to the first fastener after penetration of the bone by the first sharpened leg to enhance securement of the tendon to the bone.

17. A method for securing a tendon to a bone, the method comprising:
securing a suture to the tendon proximate a fixation site on the bone;
positioning a first fastener proximate the fixation site;
penetrating the bone of the fixation site with a first sharpened leg of the first fastener such that a capture surface of the first fastener compresses the tendon against the fixation site; and
securing the suture to the first fastener after penetration of the bone by the first sharpened leg to enhance securement of the tendon to the bone.

18. The method of claim 17, further comprising passing a first working portion of the suture through a first aperture of the first fastener, wherein securing the suture to the first fastener comprises securing the first working portion relative to the first aperture.

19. The method of claim 18, further comprising passing a second working portion of the suture through a second aperture of the first fastener, wherein securing the suture to the first fastener further comprises securing the second working portion relative to the second aperture by securing the first and second working portions together.

20. The method of claim 17, further comprising penetrating the bone of the fixation site with a second sharpened leg of the first fastener, wherein the first and second sharpened legs extend from opposite sides of the capture surface to define a channel, the method further comprising receiving the tendon within the channel.

21. The method of claim 17, further comprising:
penetrating the bone with a sharpened distal end of a second fastener; and
securing a proximal end of the second fastener to an engagement feature of the proximal portion of the first fastener.

22. The method of claim 21, wherein securing the suture to the first fastener comprises urging the first and second fasteners together to trap the suture between the first and second fasteners.

23. The method of claim 21, wherein the second fastener comprises a bone screw, wherein the proximal end comprises a head and the engagement feature comprises a hole, wherein the hole is bounded by a rim, the method further comprising:
receiving the sharpened distal end within the hole; and
seating the head against the rim such that further advancement of the sharpened distal end into the bone urges the first fastener toward the bone.

24. The method of claim 23, wherein the engagement feature comprises threads bounding the hole, wherein receiving the sharpened distal end within the hole comprises threadably receiving threads of the sharpened distal end with the threads of the engagement feature.

25. The method of claim 23, further comprising penetrating the bone of the fixation site with a second sharpened leg of the first fastener, wherein the first and second sharpened legs extend from opposite sides of the capture surface to define a channel, wherein the hole passes through the capture surface, the method further comprising:
receiving the tendon within the channel; and
advancing the second fastener through the hole to pierce the tendon with the sharpened distal end.
